(19) United States
(12) Patent Application Publication
(10) Pub. No.: US 2020/0090060 A1
(43) Pub. Date: Mar. 19, 2020

(54) SYSTEM AND METHOD FOR OPERATING A FOOD PREFERENCE ALGORITHM

(71) Applicant: NUTRISTYLE INC., Meridian, ID (US)

(72) Inventors: Scott Murdoch, Bend, OR (US); Todd Albro, Eagle, ID (US); Caleb Skinner, Beaverton, OR (US); Shannon Madsen, Livermore, CA (US); Lee Brillhart, Seattle, WA (US)

(21) Appl. No.: 16/551,552

(22) Filed: Aug. 26, 2019

Related U.S. Application Data
(60) Provisional application No. 62/723,214, filed on Aug. 27, 2018.

(51) Int. Cl. G06N 5/04; G06F 16/28; G06N 20/00; G06F 16/23; G16H 20/60

(52) U.S. Cl. CPC G06N 5/04; G06F 16/285; G16H 20/60; G06F 16/2379; G06N 20/00

(57) ABSTRACT

A method of operating a food preference algorithm involves retrieving a meal framework including at least one food component category from a meal framework database through operation of a meal selector configured by a preferences profile in a user profile, generating a meal profile including at least one food item retrieved from a food item database through operation of a food component selector configured by the meal framework and the preferences profile, operating a machine learning food preferences algorithm, and applying the updated food preferences control to the preferences profile.

### BACKGROUND

Food preferences are difficult to determine due to the many nuances associated with how different combinations of taste, texture, smell, color and previous experiences appeal to different individuals. These nuances make it difficult to generate suggestions for new recipes or dishes that other individuals may enjoy. Therefore, a need exists for improving the understanding of individual food preferences/tastes to improve food suggestions.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a system 100 in accordance with some embodiments.

FIG. 2 illustrates a method 200 in accordance with some embodiments.

FIG. 3 illustrates a system 300 in accordance with some embodiments.

FIG. 4 illustrates a process 400 in accordance with some embodiments.

FIG. 5 illustrates a system 500 in accordance with some embodiments.

FIG. 6 illustrates a system 600 in accordance with some embodiments.

FIG. 7 illustrates a system 700 in accordance with some embodiments.

FIG. 8 illustrates a system 800 in accordance with some embodiments.

FIG. 9 illustrates a system 900 in accordance with some embodiments.

### DETAILED DESCRIPTION

“Food” refers to any substance consumed to provide nutritional support for an organism. For example, food item may be assortment of consumable substances that includes meats, grains, dairy products, fruits, mushrooms, and vegetables. The food items may include condiments such as spices that may be added in combination to the aforementioned food items. Furthermore, food items may include beverages. Individual food items may be combined as components of a meal.

“Meal” refers to a single food component or combination of food components served individually or in combinations as a dish. A meal may include a dish of a variety of food components and spices accompanied by a beverage.

“Nutrient” refers to a substance used by an organism to survive, grow, and reproduce. The requirement for dietary nutrient intake applies to animals, plants, fungi, and protists. Nutrients can be incorporated into cells for metabolic purposes or excreted by cells to create non-cellular structures, such as hair, scales, feathers, or exoskeletons. Some nutrients can be metabolically converted to smaller molecules in the process of releasing energy, such as for carbohydrates, lipids, proteins, and fermentation products (ethanol or vinegar), leading to end-products of water and carbon dioxide. Nutrients include both macronutrients and micronutrients. Macronutrients provide energy and are chemical compounds that humans consume in the largest quantities and provide bulk energy are classified as carbohydrates, proteins, and fats. Water must be also consumed in large quantities. Micronutrients support metabolism and include dietary minerals and vitamins. Dietary minerals are generally trace elements, salts, or ions such as copper and iron. Some of these minerals are essential to human metabolism. Vitamins are organic compounds essential to the body. They usually act as coenzymes or cofactors for various proteins in the body.

A method of operating a food preference algorithm involves retrieving a meal or snack framework comprising at least one food component category from a meal framework database through operation of a meal selector configured by a preferences profile in a user profile, generating a meal profile comprising at least one food item retrieved from a food item database through operation of a food component selector configured by the meal framework and the preferences profile, operating a machine learning food preferences algorithm, and applying the updated food preferences control to the preferences profile.

The process of operating a machine learning food preferences algorithm involves aggregating user interactions from linked user services associated with the user profile and a meal plan menu comprising the meal profile, retrieving historic user interactions from a historic interaction database, retrieving similar user interactions from a global user interaction database, and generating an updated food preferences control from the user interactions, the historic user interactions, and the similar user interactions.

In some configurations, the preferences profile comprises food dislikes, food likes, food allergies or restrictions, meal and snack preferences (including preferred recipes), nutrient targets, weight or other personal health objectives, financial budget, preferred food brands, and grocer or food distributor preferences, kcal target and meal presets. The linked user services may comprise a food tracking service and a grocery list service.

Food preference presets may allow a dietitian or nutrition team to group foods together into meals based on the sub categories to which the foods belong. For example, one preset has the following food sub categories for a Sunday breakfast:
Egg product—Egg
Dairy—Yogurt
Beverage—water/tea
Beverage—milk, dairy, 1%
Beverage—Vegetable Juice

Each of these food sub categories contains any number of foods 1-n of which may be brand-specific. The algorithm pulls foods from each food sub category and builds menus for the users based on the user selected presets or by considering user actions.

In some configurations, a user may provide presets defining which food items to utilize for some food sub categories, identify the food item as a meal component and/or part of a larger food component category. The presets may also be utilized to identify specific food items as well as particular combinations of food items that may be viewed as individual meals by themselves.

In some configurations, a user profile may include user preferences such as food preferences (e.g., likes/dislikes, which may be further broken down into preferred tastes and/or textures, smells, etc.), restrictions (e.g., allergies or disease), health objectives (e.g., lose weight), financial budget, grocer or food distributor (e.g., grocer supplier or direct-to-consumer provider in certain food distribution scenarios), preferred brands or private labels, preferred recipes, and preferred restaurants.

The user can add foods that they like, foods they dislike, 1-n preferred brands, recipes, nutrient targets, weight or other personal health objectives, preferred grocer(s) and/or food distributor(s), and kcal target into their preferences before a food menu covering 1-n meals over 1-n days is generated for them.

The algorithm takes the preferences included in the user profile and tailors the food menu with respect to the user's specific needs/wants. The user can also add specific items that they would like to have for every meal. For instance, the user may specify that they would like an apple with every meal or a Dannon yogurt for breakfast and make sure that certain foods are never added to their specific meals.

Presets establish user specified frameworks for determining what foods are included (and excluded) from meals. In addition to establishing global preferences for food and other elements included in the user profile, the user may add increasingly granular information, such as specifying that there is a specific food and/or brand item that they would like to see appear more frequently in the meals generated by the algorithm. This includes the user being able to specify whether they want to eat the specific food item as part of every meal generated by the food menu algorithm, or whether there are specific meals (e.g., breakfast, lunch, dinner, snacks) and which days that they would like to eat those specific food items, or identify that the food can be considered when it can be purchased within financial budget guidelines. Going further, the user can specify 1-n specific macro- and/or micro-nutrient values for specific meals and/or on specific days of a multi-meal menu, for example, an elevated target for sodium or other electrolytes for meals preceding or following a strenuous run.

The food dislikes identify a specific food item and may eliminate it from being selected as a food component of any meal. In some configurations, the user may identify certain times that they would not like to eat certain food items, or when they would not like certain food items suggested. For example, a user may like tuna for lunch or dinner but may specify that they would not like to have tuna added as a food component of a meal for breakfast.

After a menu is generated a number of additional relationships may be added. These relationships pull in entities like the shopping list, food log, fitness log, restaurants, etc.

A food preference optimization algorithm may be incorporated into a meal planning system that allows the system to improve the food suggestions made to users. The algorithm may allow users to import recipes to use as part of their planned meals and then generate suggestions for other recipes based on the combinations of food items in the user's imported recipe(s). The algorithm may optimize food suggestions by identifying similar users utilizing the meal planning system, and utilizing the other user's behaviors such as swapping foods out based on their nutritional components and what foods the other users pair together to develop a taste profile for suggesting combinations of foods.

The algorithm incorporates artificial intelligence elements to enhance the accuracy of the food preference suggestions, including tracking end user behaviors to improve the predictive capability of the algorithm. For example, the algorithm may identify that when 90% of people swap a certain food item, they replace it with another specific food item. The algorithm may determine that the substitution is based on the current context and in the future avoid adding the food item to users with similar taste profiles. Additionally, the algorithm may determine that a substituted food item is highly preferred by certain users with a particular taste profile and make modifications to recipes to include that food item in the future.

In some configurations food preferences may include likes and dislikes as to food type or category, but also including taste, texture, smell, etc.

In some configurations food restrictions may include items to which the user is allergic or which are contraindicated due to a disease or because they conflict with specific nutritional supplements or medications the user is taking.

In some configurations food brands may include products “branded” by a specific food manufacturer, as well as any “private label” or “store brand” or identifier associated with the retailer, and not a supplier.

In some configurations, food distributor may include what we commonly know as a grocery store today, as well as third-party distributors who may provide certain products to grocers, but also, for example, a potential food distribution scenario in the future, where robotic warehouses fill consumer orders that are delivered by driverless vehicles. A “food distributor” could also include restaurants, food delivery services like Blue Apron, etc.

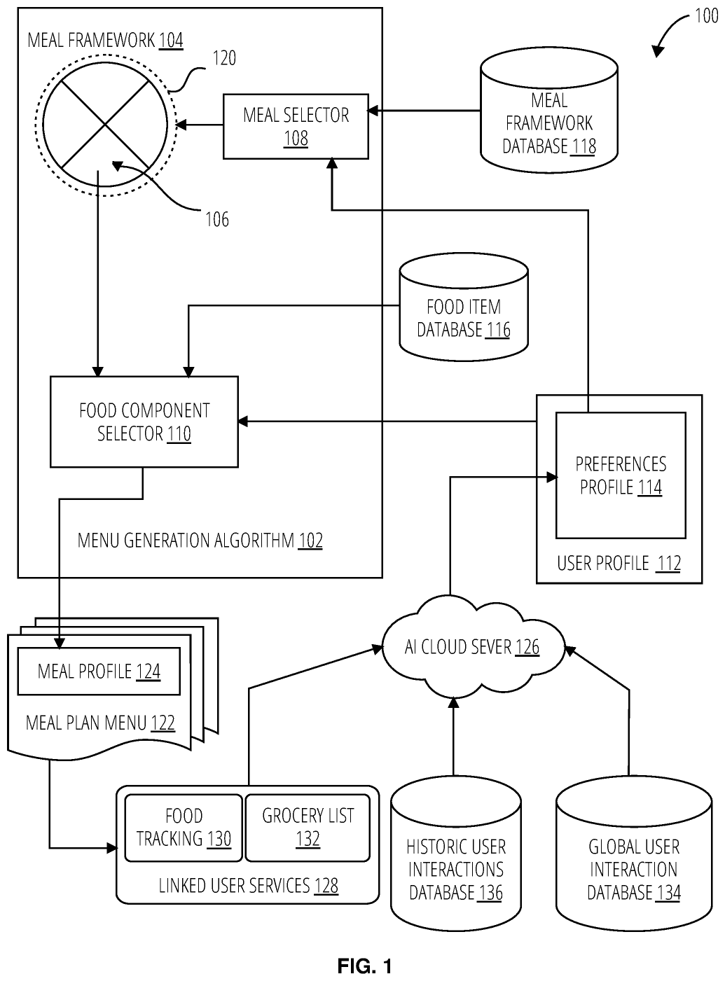

Referencing FIG. 1, a system 100 includes a menu generation algorithm 102, a meal framework database 118, a food item database 116, a linked user services 128, a historic user interactions database 136, a global user interaction database 134, and a user profile 112. The user profile 112 comprises a preferences profile 114. In some configurations, the preferences profile may include meal presets, food likes, food dislikes, food restrictions, health objectives, financial budget, brand preferences, and grocer and/or food distributor preferences. The menu generation algorithm 102 comprises a meal selector 108 and a food component selector 110. The preferences profile 114 configures a meal selector 108 to retrieve a meal framework 104 from the meal framework database 118. The selected framework meal 120 comprises at least one food component category 106. The preferences profile 114 and the at least one food component category 106 are utilized to configure a food component selector 110 to retrieve food items corresponding to the food component category 106. The food component selector 110 utilizes the food items to generate a meal profile 124 as part of a meal plan menu 122. The machine learning food preferences algorithm (AI cloud server) 126 receives information from the linked user services 128 associated with the meal plan menu 122 and the user profile 112, a historic user interactions database 136, and a global user interaction database 134. In some configurations, the linked user services 128 comprises services such as a food tracking service 130 and a grocery list service 132. The machine learning food preferences algorithm (AI cloud server) 126 utilizes the information to modify the preferences profile 114.

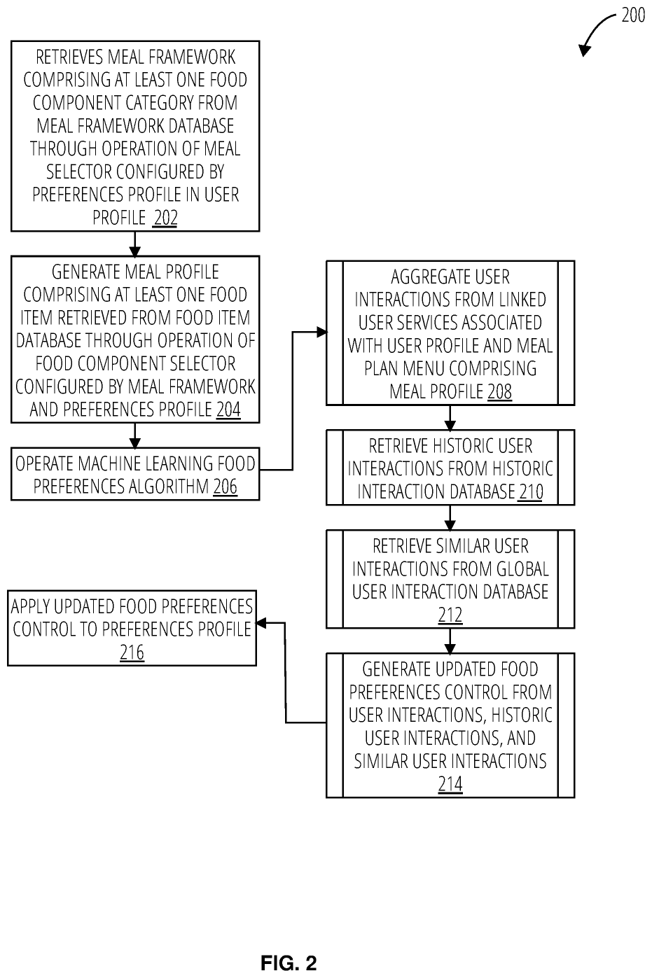

Referencing FIG. 2, a method 200 for operating a food preference algorithm includes retrieving a meal framework comprising at least one food component category from a meal framework database through operation of a meal selector configured by a preferences profile in a user profile (block 202). In block 204, the method 200 generates a meal profile comprising at least one food item retrieved from a food item database through operation of a food component selector configured by the meal framework and the preferences profile. In block 206, the method 200 operates a machine learning food preferences algorithm. In subroutine block 208, the machine learning food preferences algorithm aggregates user interactions from linked user services associated with the user profile and a meal plan menu comprising the meal profile. In subroutine block 210, the machine learning food preferences algorithm retrieves historic user interactions from a historic interaction database. In subroutine block 212, the machine learning food preferences algorithm retrieves similar user interactions from a global user interaction database. In subroutine block 214, the machine learning food preferences algorithm generates an updated food preferences control from the user interactions, the historic user interactions, and the similar user interactions. In block 216, the method 200 applies the updated food preferences control to the preferences profile.

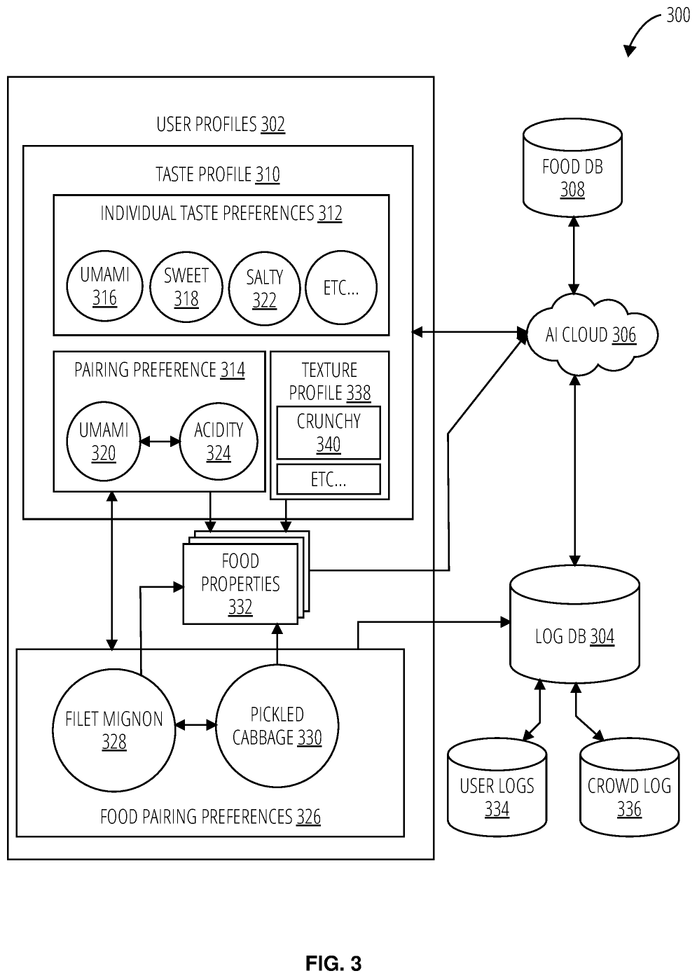

Referencing FIG. 3, a system 300 comprises user profiles 302, a log database 304, a machine learning food preferences algorithm (AI cloud) 306, and a food item database 308. The user profiles 302 comprise a taste profile 310 and food pairing preferences 326. The taste profile 310 may comprise individual taste preferences 312 including taste values for commonly accepted tastes that include, but are not limited to, umami 316, sweet 318, and salty 322. The taste profile 310 may also include pairing preference 314 including flavor pairing preference values for umami 320 and acidity 324. The taste profile 310 may also include a texture profile 338 that include, for example, a preference for crunchy 340. The food texture profile is referenced with respect to each food item, and user-specified texture or “mouth feel” preferences, determined by user input or as revealed by user activity, and is combined with the food taste profile to enhance the accuracy of the food selections included in user meals. The pairing preference 314 may be utilized in combination with established food pairing preferences 326 that may include, for example, a flavor combination for filet mignon 328 and pickled cabbage 330. The log database 304 includes information for user logs 334 and global user logs 336. The food pairing preferences 326 may record a user's food pairing preferences 326 in the user logs 334. The machine learning food preferences algorithm (AI cloud) 306 utilizes food properties 332 that are received from the food pairing preferences 326 and the pairing preference 314, the food items in the food item database 308, and logs in the log database 304 to more accurately describe the taste profile 310.

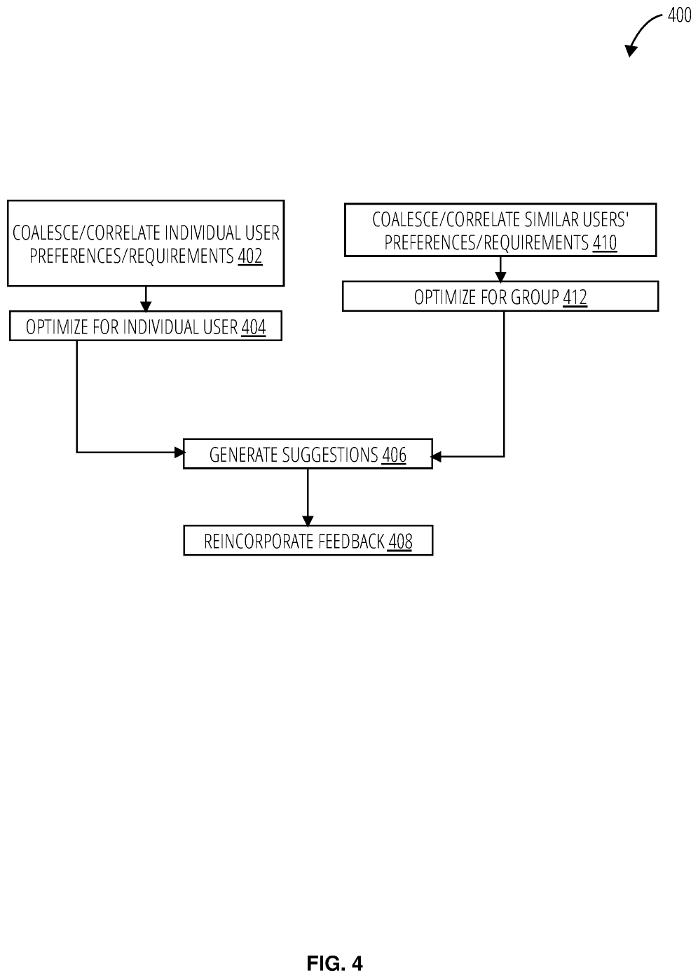

Referencing FIG. 4, a process 400 involves coalescing/correlating individual user preferences/requirements for their dietary needs (block 402). In block 404, the process 400 optimizes the food preferences for an individual user. The process 400 includes an additional branch running parallel with the branch starting with block 402. This additional branch begins with block 410, where the process 400 coalesces/correlates similar users' food preferences/requirements. The branch then continues to block 412, where the process 400 optimize food preferences for the group of similar users. Both branches meet at block 406, where the process 400 generates food item suggestions. In block 408, the process 400 reincorporates feedback to improve food item suggestions.

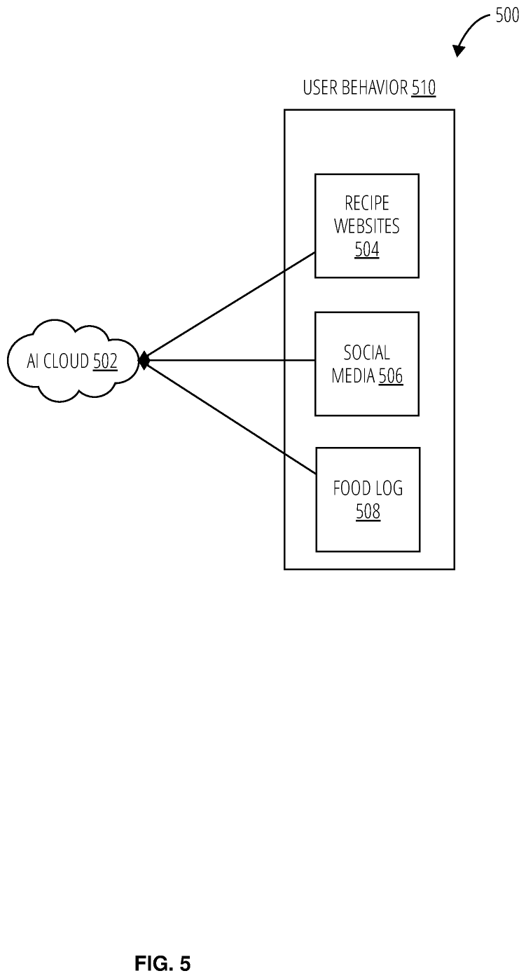

Referencing FIG. 5, a system 500 comprises a machine learning food preferences algorithm (AI cloud) 502 that utilizes user behavior 510 comprising recipe websites 504, social media 506, and a food log 508 associated with a user to improve user food preferences.

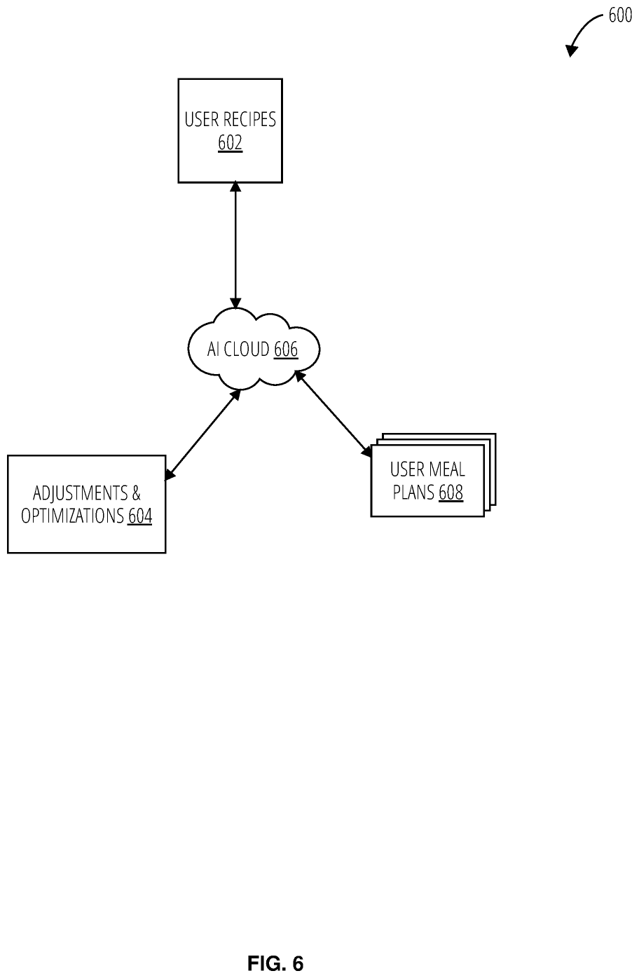

Referencing FIG. 6, a system 600 comprises a machine learning food preferences algorithm (AI cloud) 606 that utilizes user recipes 602, user meal plans 608, and user adjustments & optimizations 604 to improve user food preferences.

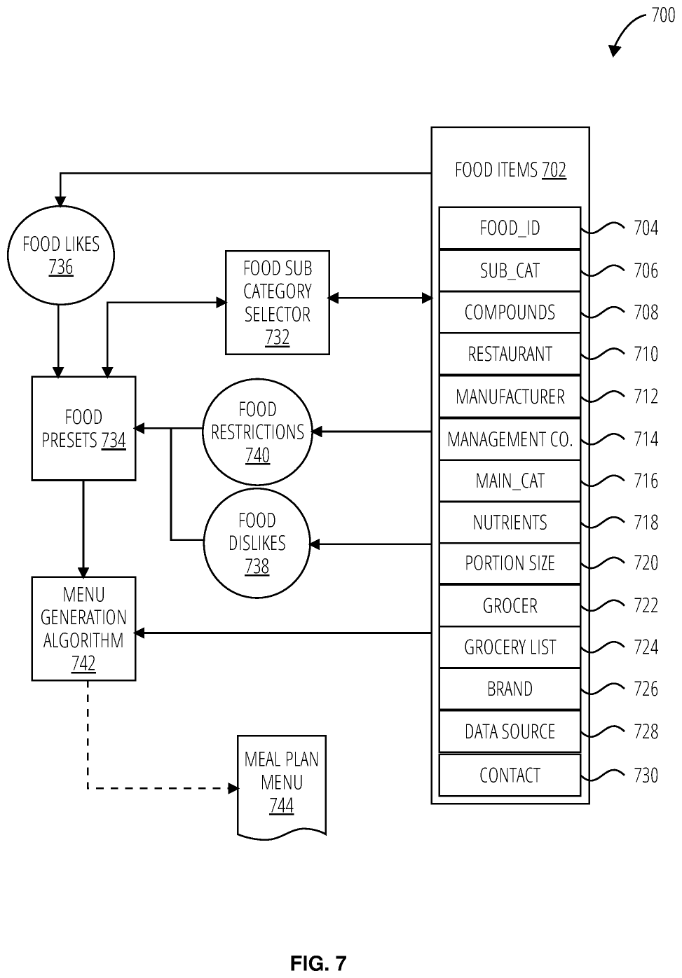

Referencing FIG. 7 a system 700 includes food items 702 comprising a food item identifier 704, a food component sub category 706, a relevant food compounds 708, a restaurant identifier 710, a food manufacturer 712, a food management company 714, a main food category 716, nutrient quantities 718, a portion size 720, a specific food distributor/grocer location 722, related grocery list information 724, a food brand name 726, a food data source 728, and food manufacturer contact information 730. A food sub category selector 732 identifies food items 702 in relevant food component sub categories based on food presets 734. The food presets 734 utilize the food restrictions 740, food dislikes 738, and the food likes 736 configured by a user to further filter food items 702 utilized in the generation of a meal plan menu 744 by the menu generation algorithm 742.

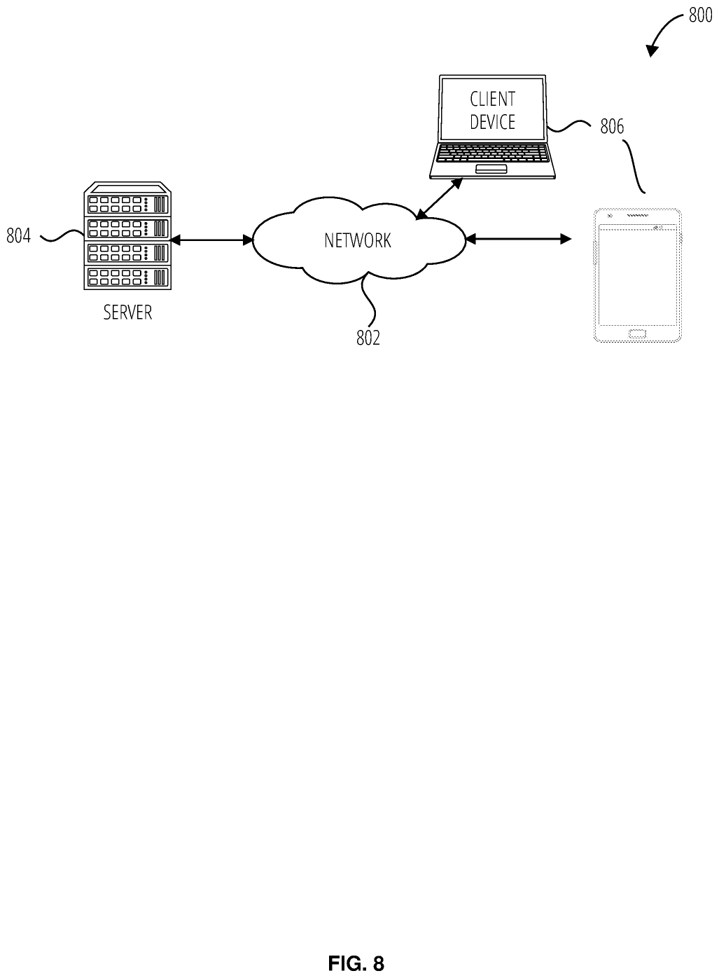

FIG. 8 illustrates a system 100 in which a server 804 and a client device 806 are connected to a network 802.

In various embodiments, the network 802 may include the Internet, a local area network (“LAN”), a wide area network (“WAN”), and/or other data network. In addition to traditional data-networking protocols, in some embodiments, data may be communicated according to protocols and/or standards including near field communication (“NFC”), Bluetooth, power-line communication (“PLC”), and the like. In some embodiments, the network 802 may also include a voice network that conveys not only voice communications, but also non-voice data such as Short Message Service (“SMS”) messages, as well as data communicated via various cellular data communication protocols, and the like.

In various embodiments, the client device 806 may include desktop PCs, mobile phones, laptops, tablets, wearable computers, or other computing devices that are capable of connecting to the network 802 and communicating with the server 804, such as described herein.

In various embodiments, additional infrastructure (e.g., short message service centers, cell sites, routers, gateways, firewalls, and the like), as well as additional devices may be present. Further, in some embodiments, the functions described as being provided by some or all of the server 804 and the client device 806 may be implemented via various combinations of physical and/or logical devices. However, it is not necessary to show such infrastructure and implementation details in FIG. 8 in order to describe an illustrative embodiment.

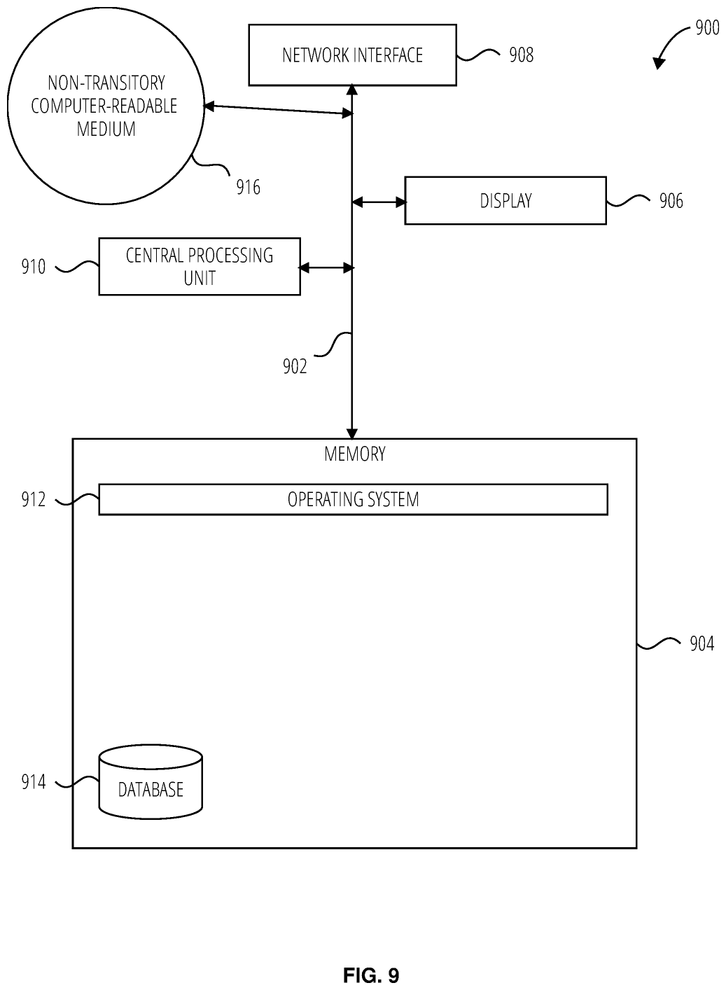

FIG. 9 illustrates several components of an exemplary system 900 in accordance with some embodiments. In various embodiments, system 900 may include a desktop PC, server, workstation, mobile phone, laptop, tablet, set-top box, appliance, or other computing device that is capable of performing operations such as those described herein. In some embodiments, system 900 may include many more components than those shown in FIG. 9. However, it is not necessary that all of these generally conventional components be shown in order to disclose an illustrative embodiment. Collectively, the various tangible components or a subset of the tangible components may be referred to herein as “logic” configured or adapted in a particular way, for example as logic configured or adapted with particular software or firmware.

In various embodiments, system 900 may comprise one or more physical and/or logical devices that collectively provide the functionalities described herein. In some embodiments, system 900 may comprise one or more replicated and/or distributed physical or logical devices.

In some embodiments, system 900 may comprise one or more computing resources provisioned from a “cloud computing” provider, for example, Amazon Elastic Compute Cloud (“Amazon EC2”), provided by Amazon.com, Inc. of Seattle, Wash.; Sun Cloud Compute Utility, provided by Sun Microsystems, Inc. of Santa Clara, Calif.; Windows Azure, provided by Microsoft Corporation of Redmond, Wash., and the like.

System 900 includes a bus 902 interconnecting several components including a network interface 908, a display 906, a central processing unit 910, and a memory 904.

Memory 904 generally comprises a random access memory (“RAM”) and permanent non-transitory mass storage device, such as a hard disk drive or solid-state drive. Memory 904 stores an operating system 912.

These and other software components may be loaded into memory 904 of system 900 using a drive mechanism (not shown) associated with a non-transitory computer-readable medium 916, such as a DVD/CD-ROM drive, memory card, network download, or the like.

Memory 904 also includes database 914. In some embodiments, system 900 may communicate with database 914 via network interface 908, a storage area network (“SAN”), a high-speed serial bus, and/or via the other suitable communication technology.

In some embodiments, database 914 may comprise one or more storage resources provisioned from a “cloud storage” provider, for example, Amazon Simple Storage Service (“Amazon S3”), provided by Amazon.com, Inc. of Seattle, Wash., Google Cloud Storage, provided by Google, Inc. of Mountain View, Calif., and the like.

Terms used herein should be accorded their ordinary meaning in the relevant arts, or the meaning indicated by their use in context, but if an express definition is provided, that meaning controls.

“Circuitry” refers to electrical circuitry having at least one discrete electrical circuit, electrical circuitry having at least one integrated circuit, electrical circuitry having at least one application specific integrated circuit, circuitry forming a general purpose computing device configured by a computer program (e.g., a general purpose computer configured by a computer program which at least partially carries out processes or devices described herein, or a microprocessor configured by a computer program which at least partially carries out processes or devices described herein), circuitry forming a memory device (e.g., forms of random access memory), or circuitry forming a communications device (e.g., a modem, communications switch, or optical-electrical equipment).

“Firmware” refers to software logic embodied as processor-executable instructions stored in read-only memories or media.

“Hardware” refers to logic embodied as analog or digital circuitry.

“Logic” refers to machine memory circuits, non transitory machine readable media, and/or circuitry which by way of its material and/or material-energy configuration comprises control and/or procedural signals, and/or settings and values (such as resistance, impedance, capacitance, inductance, current/voltage ratings, etc.), that may be applied to influence the operation of a device. Magnetic media, electronic circuits, electrical and optical memory (both volatile and nonvolatile), and firmware are examples of logic. Logic specifically excludes pure signals or software per se (however does not exclude machine memories comprising software and thereby forming configurations of matter).

“Software” refers to logic implemented as processor-executable instructions in a machine memory (e.g. read/write volatile or nonvolatile memory or media).

Herein, references to “one embodiment,” “an embodiment,” or “some embodiments” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

Various logic functional operations described herein may be implemented in logic that is referred to using a noun or noun phrase reflecting said operation or function. For example, an association operation may be carried out by an “associator” or “correlator”. Likewise, switching may be carried out by a “switch”, selection by a “selector”, and so on.

What is claimed is:

1. A method of operating a food preference algorithm, the method comprising:
retrieving a meal framework comprising at least one food component category from a meal framework database through operation of a meal selector configured by a preferences profile in a user profile;
generating a meal profile comprising at least one food item retrieved from a food item database through operation of a food component selector configured by the meal framework and the preferences profile;
operating a machine learning food preferences algorithm to:
aggregate user interactions from linked user services associated with the user profile and a meal plan menu comprising the meal profile;
retrieve historic user interactions from a historic interaction database;
retrieve similar user interactions from a global user interaction database; and
generate an updated food preferences control from the user interactions, the historic user interactions, and the similar user interactions; and
applying the updated food preferences control to the preferences profile.

2. The method of claim 1, wherein the preferences profile comprises meal presets, food likes, food dislikes, food restrictions, health objectives, kcal target, financial budget, brand preferences, and grocer and/or food distributor preferences.

3. The method of claim 1, wherein the linked user services comprise a food tracking service.

4. The method of claim 1, wherein the linked user services comprises a grocery list service.
